(19) United States
(12) Patent Application Publication
(10) Pub. No.: US 2007/0213836 A1
(43) Pub. Date: Sep. 13, 2007

(54) Surgical Ring with an Improved Closure System

(76) Inventor: Pascal Paganon, Villeurbanne (FR)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(21) Appl. No.: 10/559,200

(22) PCT Filed: Jun. 3, 2004
(86) PCT No.: PCT/FR04/01367

§ 371 Date: Feb. 8, 2007

(30) Foreign Application Priority Data
Jun. 4, 2003 (FR) 03/06790

(51) Int. Cl. A61F 2/04

(52) U.S. Cl. 623/23.64

(57) ABSTRACT

An implantable surgical ring (1, 10) for surrounding one or more organs having a pouch or a duct, in order to modify the section of the passage in the organ(s), the ring (1, 10) being in the form of a flexible strap (2, 20) extending between first and second ends (3, 4, 40, 400), the flexible strap (2, 20) being provided towards its first and second ends (3, 4, 40, 400) with respective male and female closure elements (5, 50; 6, 60, 600) arranged to co-operate in such a manner that the flexible strap (2, 20) forms a closed loop, the female closure element (6, 60, 600) permanently forming a closed collar. The female element (6, 60, 600) includes a structural discontinuity (20, 200, 2000) in which the deformability of the female element (6, 60, 600) is greater than the deformability of the remainder of the female element (6, 60, 600). The invention is applicable to implantable surgical rings.

### FIELD OF THE INVENTION

The present invention relates to the technical field of surgical implants for implanting in the body of a patient around one or more organs constituting a pouch or a duct, and more particularly it relates to gastric rings for treating obesity by implanting a flexible gastric ring that forms a closed loop around the stomach in order to reduce the diameter of the stoma opening.

The present invention relates to an implantable surgical ring for surrounding one or more organs having a pouch or a duct, in order to modify the section of the passage of said organ, said ring being in the form of a flexible strap extending between first and second ends, said flexible strap being provided towards its first and second ends with respective male and female closure elements arranged to co-operate in such a manner that the flexible strap forms a closed loop, the female closure element permanently forming a closed collar.

More particularly, the present invention relates to a gastroplasty ring, however it can also relate to a ring designed to be used for treating urinary or fecal incontinence (artificial sphincter), or indeed a ring designed to control blood flow in blood vessels, for example, this list not being limiting in any way.

### BACKGROUND OF THE INVENTION

It is already known to perform surgery on patients suffering from extremely severe obesity (morbid obesity), i.e. for patients whose weight exceeds the ideal weight by at least 50 kilograms (kg), by implanting a gastroplasty ring in the body of such a patient.

Such surgery makes it possible not only to avoid serious health problems due to overweight, but also and above all, to avoid certain and imminent death of such patients.

It is accepted that patients suffering from morbid obesity have their life expectancy reduced very considerably, generally by at least 10 to 15 years, while also suffering from considerable problems of psychological load.

Furthermore, an entire series of associated health phenomena is implicated, having an effect on the occurrence of associated diseases, such as cardiovascular disease, hypertension, diabetes, or indeed severe arthritis, in particular.

It is also accepted that with such patients, treatments based on severe diet combined with a series of physical exercise, also associated with a change in behavior, in particular in eating habits, are generally of little effect, even though such methods of treatment are recognized as being the most healthy.

That is why effective and long-term treatments of morbid obesity make use of surgical treatment.

In general, a distinction is drawn between surgical treatment techniques that involve reducing food absorption, i.e. shortening the path followed by food and digestive juices, and techniques that make use of gastric restriction to reduce the size of the stomach.

Techniques that involve reducing food absorption are those involving, for example, a technique of bypassing the small intestine, or indeed techniques that establish a separation between the path followed by food and that followed by digestive juices.

Those surgical techniques are relatively burdensome and can give rise to severe complications, which is why they are used very little nowadays.

The present trend is to use surgical techniques that make use of smaller amounts of surgery, such as gastric restriction that involves putting a gastric ring into place.

These techniques are now in quite widespread use, and for the most part (and as described for example in document SE-449 430) they make use of a flexible strap for implanting around the stomach, so as to form a loop that is closed by means of a closure system and that defines an unvarying perimeter for the ring.

Against its surface that is to come into contact with the stomach, the body of the flexible strap includes a variable-volume inflation chamber that is connected to an adjustable catheter enabling fluids to be injected into or withdrawn from the inflation chamber so as to vary the internal perimeter of the loop in order to modify or adjust the diameter of the stoma.

Thus, in combination with the unvarying and preestablished diameter of the ring, it is possible to adjust the diameter of the ring to a relatively small extent, thus enabling the diameter of the stoma to be adjusted, and thus enabling the quantity of food that is ingested to be adjusted.

The ring closure system described in document SE-449 430 implements male/female coupling between a head that forms a male member secured to a first end of the flexible strap, and provided with a series of deformable indentations, and a female housing of complementary shape, secured to the second end of the flexible strap, said head being arranged to be forced into the female housing and come into latching co-operation therewith. Said latching co-operation thus closes the ring to form a closed loop.

At least in terms of its general principles, that closure system is generally appreciated by surgeons since the closure operation takes place in continuity with circling the stomach, and in the same direction as the circling. The surgeon can thus perform the complete operation of making a closed loop around the stomach by actions that are substantially continuous.

Nevertheless, such a closure system can present certain drawbacks.

Thus, the head must be made of a material that is relatively rigid, in order to guarantee that the ring is closed in stable manner.

However that rigidity puts the limit on the size of the indentations since otherwise it is necessary to apply an extremely large force, e.g. significantly greater than 15 newtons (N), in order to force the head into the female housing so as to close the ring. Unfortunately, a large force is harmful to the components of the rings themselves, since they can be damaged under the effect of such stress, and it is also harmful to the operation proceeding smoothly since requiring a surgeon to apply high levels of force generally reduces the precision of the surgeon's actions, and leads to a greater risk of accident.

However, making indentations that are small in size is harmful to the stability of ring closure, increasing the risk of the head becoming separated in untimely manner from the female housing.

The compromise between the force required for closure, and the reliability of said closure, and also the cost of the ring therefore turns out not to be optimized at present.

Furthermore, the problem associated with this compromise is particularly significant when the ring is provided with an actuator for adjusting the internal perimeter of the ring, and when the actuator is based on an electric motor instead of on a pneumatic system, as described in document SE-449 430.

Such an actuator generally presents a relatively large volume, which prevents it from being used as the male closure element. Thus, even when miniaturized, such an actuator when used as the male element requires the surgeon to exert too much force in order to force the actuator through a corresponding female housing.

In addition, such an actuator presents the characteristic of being generally rigid, which lends itself poorly to implementing a male/female closure system that requires the male element to be passed by force with elastic deformation into the female element.

Thus, on the basis of the teaching of document SE-449 430, it appears to be out of the question to make a male/female closure system when the ring is provided with an actuator of the electric motor type.

### SUMMARY OF THE INVENTION

Consequently, the invention seeks to provide, in one aspect, a remedy to the various drawbacks listed above, and to propose a novel surgical ring, in particular a gastric ring, that presents a male/female type closure system that is particularly reliable, and that does not require a large amount of effort on the part of the surgeon in order to operate it, even when the male element is bulky.

Another feature of the invention is to propose a novel surgical ring, in particular a gastric ring, in which the closure system can be activated and deactivated at will.

Another feature of the invention is to propose a novel surgical ring, in particular a gastric ring, in which implementation is particularly practical for the surgeon.

Another feature of the invention is to propose a novel surgical ring, in particular a gastric ring, presenting a closure system that is particularly reliable.

Another feature of the invention is to propose a novel surgical ring, in particular a gastric ring, presenting a closure system that is particularly stable.

Another feature of the invention is to propose a novel surgical ring, in particular a gastric ring, which is particularly compact and easy to manufacture.

Another feature of the invention is to propose a novel surgical ring, in particular a gastric ring, which is easy to manipulate both while being put into place and while being opened or closed.

Another feature of the invention is to propose a novel surgical ring, in particular a gastric ring, in which the closure system is particularly well adapted to the presence of an actuator for varying the internal perimeter of the ring.

These features are achieved by means of an implantable surgical ring for surrounding one or more organs having a pouch or a duct, in order to modify the section of the passage in said organ(s), said ring being in the form of a flexible strap extending between first and second ends, said flexible strap being provided towards its first and second ends with respective male and female closure elements arranged to co-operate in such a manner that the flexible strap forms a closed loop, the female closure element permanently forming a closed collar, the ring being characterized in that said female element includes a structural discontinuity in which the deformability of the female element is greater than the deformability of the remainder of the female element.

### BRIEF DESCRIPTION OF THE DRAWINGS

Other features and advantages of the invention appear better on reading the following description and with the help of the accompanying drawings that are given solely for illustration and for information, and in which:

FIG. 1 is a fragmentary perspective view showing an example of a first variant exemplary embodiment of a gastric ring in accordance with the invention, when the ring is in the open position, i.e. when it does form a closed loop;

FIG. 2 is a perspective view of the FIG. 1 gastric ring in the non-locked closed position, said ring being fitted with a reception antenna;

FIG. 3 is a fragmentary perspective view showing the male and female closure elements of the gastric ring of FIGS. 1 and 2, when the ring is locked in the closure position;

FIG. 4 is a fragmentary perspective view showing the female closure element of the gastric ring shown in FIGS. 1 to 3;

FIG. 5 is a fragmentary perspective view showing co-operation between the female closure element shown in FIG. 4 and a male element, while the male element is being inserted into the female element;

FIG. 6 is a perspective view showing an example of a second variant exemplary embodiment of a gastric ring in accordance with the invention, said gastric ring being in the closure position, but not locked;

FIG. 7 is a perspective view showing an exemplary embodiment detail of a third variant exemplary embodiment of a gastric ring in accordance with the invention, said detail acting as a female closure element for said gastric ring;

FIG. 8 is a perspective view showing the FIG. 7 female element while it is in its configuration for passing the male element;

FIG. 9 is a section view showing an exemplary embodiment detail of the FIG. 2 gastric ring;

FIG. 10 is a plan view showing the female closure element of a gastric ring in accordance with a fourth exemplary embodiment of the invention, said ring being locked in the closure position; and

FIG. 11 is a plan view showing the FIG. 10 female closure element with the ring being in the closure position, but unlocked.

### DESCRIPTION OF THE INVENTION

In the description below, reference is made purely by way of example to a gastric ring designed to be implanted around the stomach in order to reduce the diameter of the stoma opening, or else around the esophagus.

Nevertheless, the invention is not limited in any way to this application, and on the contrary it seeks to cover in general manner any implantable surgical ring for surrounding one or more organs constituting a pouch or a duct, in order to modify the section of the passage in said organ.

The invention can thus apply in particular to the rings used for treating urinary or fecal incontinence, or used around blood vessels for controlling blood flow, for example. In treatment for urinary incontinence, the ring is implanted around the bladder or the urinary tract, and for fecal incontinence, it is implanted around the gastrointestinal tract, in particular around the anal structures of the intestine.

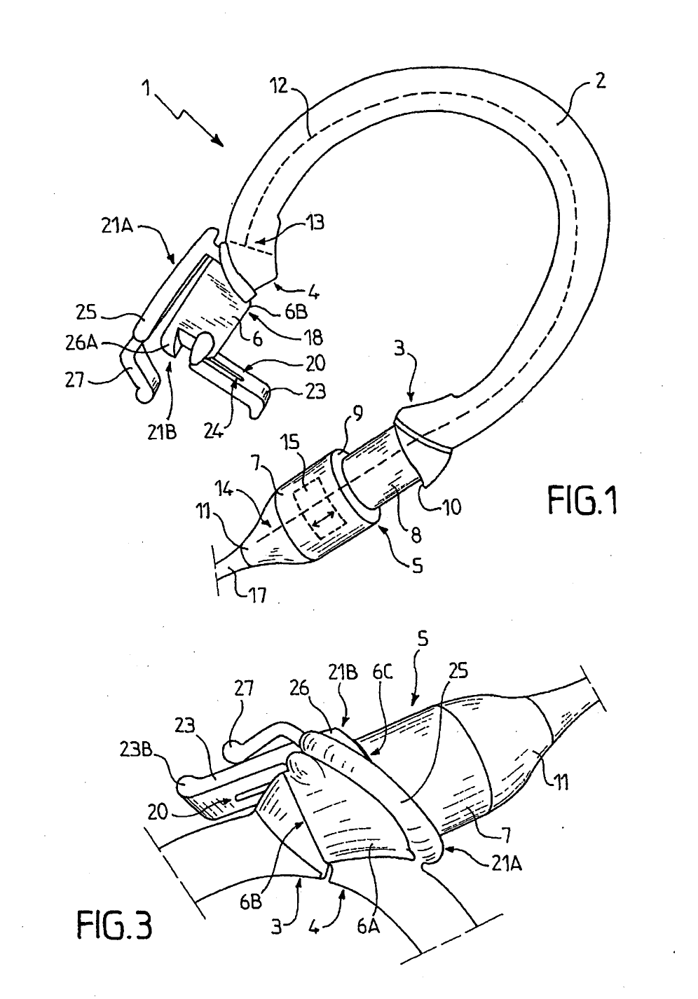

FIGS. 1 to 10 show a gastric ring 1, in accordance with the invention for implanting around the stomach of a patient in the form of a substantially circular loop, in order to implement gastric restriction by reducing the diameter of the opening of the stoma.

The gastric ring 1, in accordance with the invention is in the form of a flexible strap 2, 20, said flexible strap preferably being a flexible tubular element having a flexible and elastic skin that presents a surface that is smooth so as to be non-traumatic, so as to enable it to be well tolerated by the patient and by stomach tissues.

The flexible strap 2, 20 extends between a first end 3 and a second end 4, 40, 400.

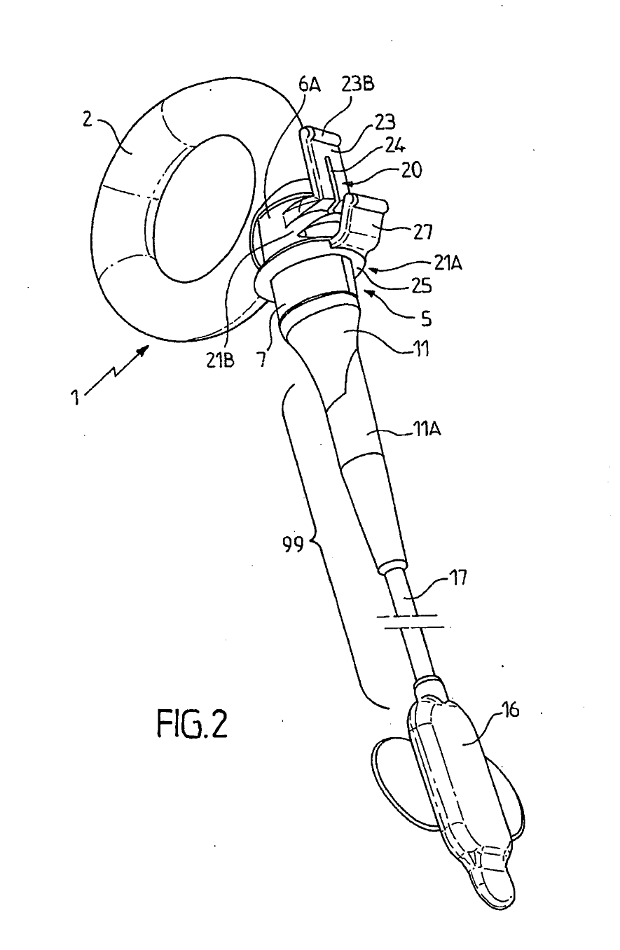

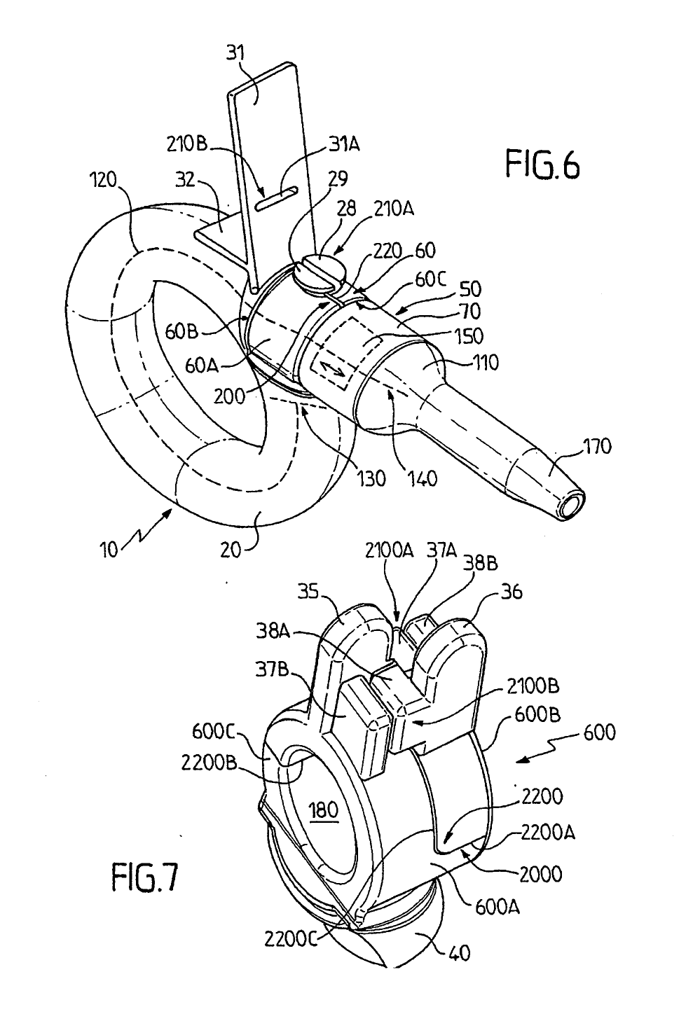

Towards its first end 3, the flexible strap 2, 20 is provided with a male closure element 5, 50. Towards its second end 4, 40, 400, the flexible strap 2, 20 is also provided with a female closure element 6, 60, 600. Said male and female closure elements 5, 50, 6, 60, 600 are arranged to co-operate so as to close the flexible strap 2, 20 around the stomach and thus constitute a closed loop between the two ends 3, 4, 40, 400 as shown in FIGS. 2 and 6, for example.

Once closed, the gastric ring 1, in accordance with the invention is then substantially in the form of a torus of revolution, e.g. of circular section, defined on the outside by a single-layer or multi-layer skin that may advantageously be formed by a protective covering, e.g. based on or made of silicone.

As can be seen in particular in FIGS. 1 to 3 and 6, the male closure element 5, 50 comprises at least a first portion 7, 70 and a second portion 8, with the cross-section of the first portion 7, 70 being substantially greater than that of the second portion 8. In other words, the first portion 7, 70 is significantly bulkier than the second portion 8.

Preferably, the male element 5, 50 is designed for having its first portion 7, 70 engaged by force in the female element 6, 60, 600 until the female element 6, 60, 600 co-operates by closing engagement with the second portion 8 of the male element 5, 50. Said second portion 8 is thus designed to be interposed between the first portion 7, 70 and the first end 3 of the flexible strap 2, 20. The first end 3 is thus extended by the second portion 8 which is in turn extended by the first portion 7, 70.

In order to make it easier to engage the male element 5, 50 in the female element 6, 60, 600, the end of the first portion 7, 70 that is to be inserted first into the female element 6, 60, 600 is advantageously extended by a converging shape 11, 110, e.g. a shape that is substantially frustoconical.

Preferably, the male element 5, 50 presents a shape that is substantially cylindrical, e.g. of circular section, said cylinder being provided with a groove set back from its periphery, said groove forming the second portion 8.

The junction between the first portion 7, 70 and the second portion 8 is then preferably implemented by means of a first shoulder 9 forming a plane annular surface. The second portion 8 thus extends between said first shoulder 9 and a second shoulder 10 between the second portion 8 and the remainder of the flexible strap 2, said second shoulder 10 being situated at the first end 3.

In the context of the invention, the first portion 7, 70 and/or the second portion 8 may be substantially rigid. In particular, in the context of the invention, it is not essential for the first portion 7, 70 of the male element 5, 50 to be deformable.

As shown in particular in FIGS. 1 and 6, the gastric ring 1, in accordance with the invention advantageously includes a system for reversibly controlling variation in its internal perimeter. Said system preferably comprises an elongate flexible element 12, 120 presenting good flexibility and good mechanical strength, said elongate flexible element 12, 120 being inserted longitudinally and sliding along the main axis of symmetry of the cylinder constituting the main body of the ring 1.

As shown in particular in FIGS. 1 and 6, said elongate element 12, 120 occupies a cavity interconnecting the first and second ends 3, 4, 40, 400 and extends substantially between said first and second ends 3, 4, 40, 400, i.e. substantially along the entire developed length of the ring 1.

The elongate flexible element 12, 120 is mounted so as to define a stationary portion 13, 130 which is secured by appropriate means, e.g. involving a clip and a washer, to the second end 4, 40, 400 of the ring 1.

The other terminal portion of the elongate flexible element 12, 120 forms a free portion 14, 140, i.e. a portion that can be moved in translation relative to the stationary portion 13, 130. Said free portion 14, 140 is functionally associated with an actuator 15, 150 mounted on the ring 1, towards or at its first end 3.

On being actuated, the actuator 15, 150 serves to transmit the energy needed to move the elongate flexible element 12, 120 in reversible translation relative to the stationary portion 13, 130 in order to obtain an associated change in the perimeter of the ring 1, i.e. an increase or a decrease in its diameter. In preferred manner, the actuator 15, 150 comprises an electric motor.

Mounting the actuator 15, 150 directly at one of ends 3 of the ring 1 thus enables a significant saving in space to be achieved and also provides good mechanical effectiveness.

Advantageously, the actuator 15, 150 is arranged on the flexible strap 2 in such a manner as to constitute and/or be included in the first portion 7, 70 of the male element 5, 50. In even more preferred manner, the actuator 15, 150 is received inside a cylindrical cavity 7A (cf. FIG. 5) formed within the first portion 7, 70, which itself presents a shape that is generally cylindrical and of circular section.

Advantageously, the elongate flexible element 12, 120 is provided with means for providing forced co-operation with the actuator, said means for providing forced co-operation advantageously being constituted by a screw thread. For this purpose, the elongate flexible element 12, 120 is constituted, for example, by a flexible core that is preferably made of metal, e.g. of circular section, having at least one spring secured thereto and wound coaxially thereabout, e.g. over its entire length, the spring(s) having non-touching turns forming the screw thread(s).

The gastric ring 1, in accordance with the invention is particularly, but not exclusively, designed to be integrated in a system for remotely restricting and controlling the ingestion of food in the stomach of a patient, in such a manner as to be able to vary the diameter of the ring 1, remotely and without any invasive surgery.

For this purpose, the actuator-forming electric motor 15, 150 is advantageously connected to a subcutaneous receiver circuit provided with a receiver antenna 16 (cf. FIG. 2) for receiving a radiofrequency (RF) control and power signal, the assembly being designed to be implanted in the body of the patient.

The electric motor 15, 150, which does not have any internal power supply, is functionally connected via an electrical connection 17A to the circuit of the receiver antenna 16. This electrical connection 17A is itself coated in a protective catheter 17, e.g. made of silicone.

Advantageously, the converging shape 11 is extended by a tapering sheath 11A containing a portion of the protective catheter 17. The tapering sheath 11A, like the shape 11, is preferably made of a material presenting low hardness, e.g. silicone having hardness equal to 40 on the Shore A scale. Selecting such a low-hardness material for making the tapering sheath 11A enables the sheath to be penetrated internally by the free end 14 of the elongate flexible element 12 when it is moved under drive from the motor 15 (see FIG. 9).

Nevertheless, for making the protective catheter 17, it is preferred to use a material presenting hardness greater than that of the material constituting the tapering sheath 11A. By way of example, the protective catheter 17 may be made of silicone having hardness equal to 80 on the Shore A scale.

The combination of a hard catheter 17 with a tapering sheath 11A of smaller hardness overmolded thereon enables an excellent compromise to be found between flexibility, rigidity, and strength for this zone 99 of the ring 1 that extends between the male element 5 and the receiver antenna 16. This compromise enables flexible contact to be imparted to the zone 99 suitable for limiting, or even eliminating any aggression to tissue close to the stomach, while nevertheless, by means of the presence of a hard catheter, providing protection against possible flattening coming from the celioscope forceps used by the surgeon to take hold of the zone 99 and pull said zone in order to close the ring 1. The presence of a tapering sheath 11A of small hardness also makes it easier for such celioscope forceps to grasp it because of its soft nature.

Nevertheless, without going beyond the ambit of the invention, it is entirely possible to envisage the ring 1, being provided with a diameter-varying system that is implemented using pneumatic means, e.g. by injecting or withdrawing fluid by means of the catheter 17, which then acts as a fluid duct between a source of fluid (connected in the position of the antenna 16) and an inflation chamber formed within the flexible strap 2.

It is also entirely possible to envisage the ring 1 having no means for varying its diameter.

In either of these two circumstances, the first portion 7, 70 no longer performs any function of housing or protection for any actuator means, but merely performs its role as a male closure element.

In accordance with an important characteristic of the invention, the female closure element 6, 60, 600 is designed to vary in shape by deforming elastically between a resilient return configuration forming its closure configuration and a male element passing configuration, and preferably to do so in reversible manner.

The term “reversible” is used herein to mean that the female closure element can be caused to pass at will from its closure configuration to its male element passing configuration (and vice versa) without suffering any irreversible structural damage, such as being destroyed.

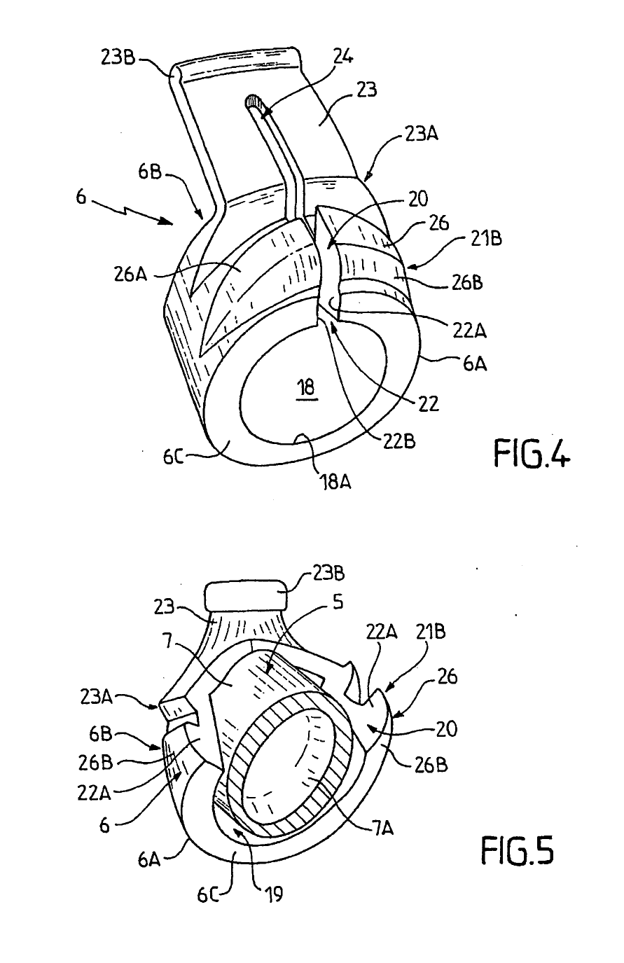

As shown in particular in FIGS. 4 and 7, the closure configuration comprises a configuration in which the female element 6, 60, 600 forms and defines a small opening 18, 180 through which the second portion 8 of the male element 5, 50 can pass but that prevents or at least opposes passage of the first portion 7, 70 of the male element 5, 50.

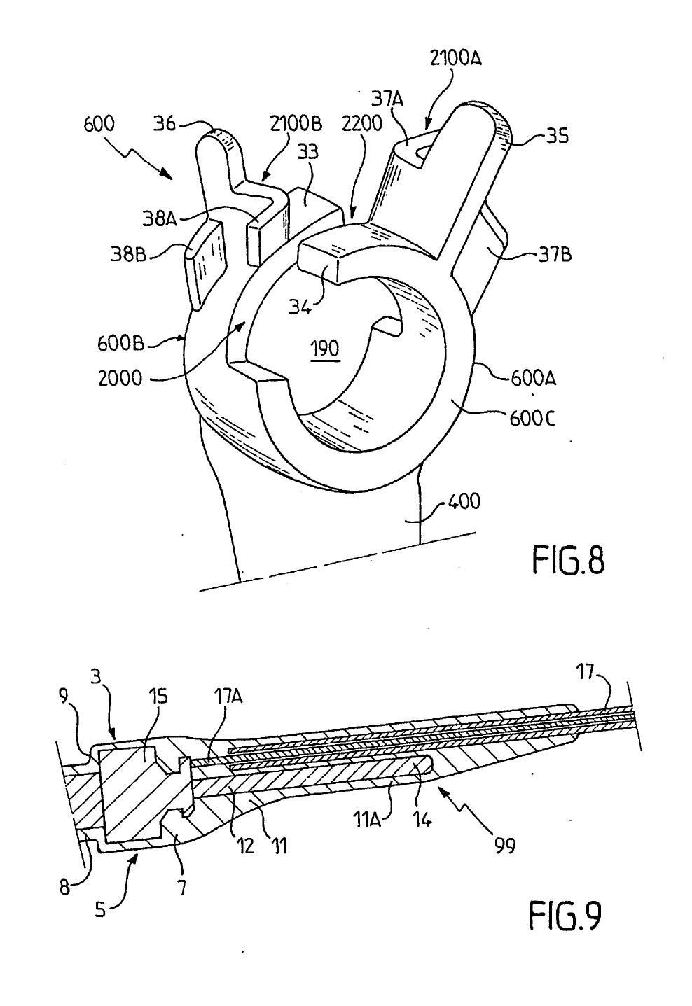

In contrast, when the female element 6, 60, 600 is in the male element passing configuration, as shown for example in FIGS. 5 and 8, it forms an enlarged opening 19, 190 allowing the first portion 7, 70 of the male element 5, 50 to pass through.

In other words, the female element 6, 60, 600 is capable of being distended from a closure configuration to an opening configuration, said distension preferably being caused by forcing the first portion 7, 70 through the opening 18, 180 defined by the female element 6, 60, 600 even though the section of the first portion 7, 70 is greater than that of the opening 18, 180 while the female element 6, 60, 600 is in the closure configuration. When the stress caused by said forced passage of the first portion 7, 70 disappears, then the female element 6, 60, 600 returns automatically by elastic return or more generally by shape memory to its closure configuration, which closure configuration is shown in particular in FIGS. 1 to 4, 6, and 7.

The first portion 7, 70 thus forms an abutment of dimensions suitable for presenting a cross-section that is greater than that of the small opening 18, 180, such that the ring is closed by pressing the female element against said abutment.

In accordance with an important characteristic of the invention, the female element 6, 60, 600 includes a structural discontinuity 20, 200, 2000 where the deformability of the female element 6, 60, 600 is greater than the deformability of the remainder of the female element 6, 60, 600.

The term “structural discontinuity” is used herein to mean that the structure of the female element 6, 60, 600 can vary to a considerable extent in three dimensions, and preferably suddenly, although such variation could also be relatively progressive.

In the context of the invention, a structural discontinuity may thus be constituted by a change in the mechanical properties of the material forming the female element 6, 60, 600, with this significant change in properties being the result, for example, of a change in the composition of the material constituting the female element, or indeed by creating empty zones in the female element 6, 60, 600, i.e. zones having no material, i.e. zones that are “infinitely” deformable.

In accordance with the invention, the structural discontinuity 20, 200, 2000 is thus achieved in such a manner that the capacity of the female element 6, 60, 600 for deforming is greater in said discontinuity 20, 200, 2000 than substantially anywhere else in the remainder of the female element 6, 60, 600.

In other words, when the female element 6, 60, 600 is subjected to a given level of mechanical stress, the female element 6, 60, 600 tends to deform substantially more easily in the structural discontinuity 20, 200, 2000 than in other zones belonging to said female element 6, 60, 600.

The female element 6, 60, 600 thus presents, in three dimensions, a gradient in its ability to deform, or more simply it presents deformation ability that is substantially constant except in certain singular zones corresponding to the structural discontinuity 20, 200, 2000.

Deformation of the female element 6, 60, 600, i.e. a change in the dimensions of said female element 6, 60, 600, and/or a change in its shape, thus requires a mechanical stress to be applied at a level that is generally lower than the mechanical stress needed for obtaining the same changes in dimension and/or shape in the absence of the structural discontinuity 20, 200, 2000.

This particular disposition thus makes it easy for the first portion 7, 70 to pass through the female element 6, 60, 600 since the presence of a zone of weakness, i.e. a zone of greater flexibility and reduced rigidity, forming the structural discontinuity 20, 200, 2000, makes it possible to cause the female element 6, 60, 600 to pass into its male element passing configuration without it being necessary to exert a force that is as great as the force that would need to be exerted if the deformability of the female element 6, 60, 600 were constant throughout.

The invention thus makes it possible to benefit from a female element 6, 60, 600 that simultaneously presents the nature of being rigid and difficult to deform, thus enabling closure to be stable and minimizing any risk of untimely opening, and also a capacity to pass from the closure configuration to the opening configuration in relatively easy manner because of the presence of a structural deformation that is easier to deform than the remainder of the female element 6, 60, 600.

Advantageously, the ring 1, includes means 21A, 21B, 210A, 210B, 2100A, 21 φB for locking and unlocking the female closure element 6, 60, 600 in its closure configuration. The locking/unlocking means 21A, 21B, 210A, 210B, 2100A, 2100B thus make it possible at will to block the female closure element 6, 60, 600 in its locking configuration, i.e. to prevent the female closure element 6, 60, 600 from passing into its male element passing configuration, even in the event of untimely mechanical stress being applied thereto.

The locking/unlocking means 21A, 21B, 210A, 210B, 2100A, 2100B may naturally be activated and deactivated at will.

Advantageously, the locking/unlocking means 21A, 21B, 210A, 210B, 2100A, 21 φB are arranged to vary between a locking position (shown in FIGS. 3 and 7 for example) in which they inhibit the deformability at the structural discontinuity 20, 200, 2000, and an unlocking position, e.g. as shown in FIGS. 1, 2, and 6 where they substantially release the female closure element 6, 60, 600 from an locking stress.

When in the locking position, the locking/unlocking means 21A, 21B, 210A, 210B, 2100A, 2100B are thus capable substantially of preventing or at least greatly reducing the ability of the structural discontinuity 20, 200, 2000 to deform.

Preferably, the locking/unlocking means 21A, 21B, 210A, 210B, 2100A, 2100B are functionally distinct from the closure elements 5, 50, 6, 60, 600 so that the locking/unlocking operation is independent of the closure operation.

Advantageously, the female element 6, 60, 600 comprises a tube, said tube itself having a side wall 6A, 60A, 600A that is preferably generally cylindrical in shape, extending longitudinally between a first face 6B, 60B, 600B and a opposite second face 6C, 60C, 600C. Said side wall 6A, 60A, 600A defines the small opening 18, 180 when the tube 6, 60, 600 is in the closure configuration. The male element 5, 50 is for engaging in the opening in the tube 6, 60, 600 going from the first face 6B, 60B, 600B towards the second face 6C, 60C, 600C.

Advantageously, the side wall 6A, 60A, 600A is split at least by a first slot 22, 220, 2200 such that said first slot 22, 220, 2200 forms said structural discontinuity 20, 200, 2000.

Preferably, said first slot 22, 220, 2220 is formed through the entire thickness and the entire length of the side wall 6A, 60A, 600A, said first slot 22, 220, 2200 possibly nevertheless being made in some other way, so as to achieve the desired function of greater ability to deform.

Below, three alternative exemplary embodiments of said first slot 22, 220, 2200 are described in succession, it being understood that other exemplary embodiments are also possible, and in particular instead of said slot 22, 220, 2200, it is possible to provide a material presenting stiffness that is different from (i.e. less than) the stiffness of the material forming the remainder of the female element 6, 60, 600.

In the first variant exemplary embodiment shown in FIGS. 1 to 5, the female closure element comprises a tube 6 that is generally in the shape of a right circular cylinder.

The side wall 6A forming the body of the tube 6 thus extends longitudinally between first and second opposite faces 6B and 6C.

The first slot 22 preferably extends between the first and second faces 6B and 6C in longitudinal and rectilinear manner, said first slot 22 opening out into each of said faces 6B and 6C.

The first slot 22 thus extends over the entire length of the tube, and through the entire thickness of the wall 6A.

The tube 6 is thus formed by a strip of preferably elastomeric material that is rolled up to define a central opening 18, with each of the ends of said strip of material forming a respective lip 22A, 22B defining the first slot 22.

The tube 6 is preferably made out of a single elastic material which is preferably constituted by or based on silicone.

The section of the small opening 18 defined by the tube 6 when the tube is in its rest position, i.e. when it is not being subjected to external mechanical stress, substantially matches the section of the second portion 8. In other words, the generally circular outline 18A defining the opening 18 is of a diameter that is slightly greater than or substantially equal to the diameter of the cylindrical portion forming the second portion 8.

Without thereby going beyond the ambit of the invention, it is nevertheless entirely possible to envisage the tube 6 defining a small opening 18 of section that is not circular but that is of any other shape, e.g. rectangular. Under such circumstances, the shape of the cross-section of the second portion 8 should preferably match and be complementary to that of the small opening 18.

Advantageously, the tube 6 is provided with a first pull-tab 23 arranged to make it easier to cause the male element 5 and the tube 6 to move towards each other and enter into co-operation and/or to make it easier for said male and female elements 5 and 6 to be separated and moved apart.

The first pull-tab 23 is preferably attached to the side wall 6A of the tube 6, projecting outwards from the closed loop formed by the flexible strap 2 (see FIG. 2), so as to make it easier to move the tube 6 and the male element 5 towards each other or apart from each other, and also so as to facilitate mutual connection and disconnection of said male and female elements 5 and 6.

Advantageously, the first pull-tab 23, which preferably comprises as its main body a substantially plane rectangular tongue made of elastic material, extends between a first attachment end 23A secured to the tube 6 and a free end 23B for being grasped by the surgeon using forceps.

More particularly, the attachment end 23A is secured to the side wall 6A of the tube 6 close to the second face 6C of the tube 6. Said first pull-tab 23 extends substantially tangentially to the outside of the side wall 6A in a direction that is substantially parallel to the longitudinal direction of the side wall 6A of said tube 6.

Preferably, the first pull-tab 23 is split longitudinally throughout its thickness by a second slot 24, which second slot 24 preferably extends over a fraction only of the length of the tab 23 from its attachment end 23A (cf. FIG. 4). Said first pull-tab 23 is also attached to the tube 6 in the vicinity of the first slot 22 so that said first slot 22 is extended by the second slot 24, said first and second slots 22 and 24 communicating to form a single resulting slot that extends from the first face 6B of the tube 6.

Because this single resulting slot 22, 24 does not go right through the first pull-tab 23, i.e. does not open out into its end 23B for grasping, the tube 6 can be maintained in relatively stable and rigid form, thus permanently forming a closed collar (i.e. presenting continuity of material in its open configuration and in its passage configuration) but of variable diameter, while being split preferably in part only so as to allow and facilitate insertion of the male element 5.

Nevertheless, it is entirely possible, without going beyond the ambit of the invention, to envisage the second slot 24 extending over the entire length of the first pull-tab 23, all the way to its end 23B for grasping.

As shown in FIGS. 1 to 5, the first variant exemplary embodiment is advantageously provided with locking/unlocking means 21A, 21B for the female closure element 6 when in its closure configuration. Said locking/unlocking means 21A, 21B preferably comprises firstly a flexible band 25 and secondly a latching ridge 26.

The flexible band 25 is of annular structure and it is preferably made of a material that is elastic, or that at least presents a certain amount of flexibility. The flexible band 25 is preferably secured to the remainder of the ring 1 via the second end 4 of the ring 1.

Advantageously, and as shown for example in FIGS. 1 to 3, the flexible band 25 is locally attached to the flexible strap 2 and/or to the tube 6 so that said band 25 extends substantially in a plane situated in front of and close to the second face 6B of the tube 6.

The latching ridge 26 is situated on the outer periphery of the tube 6, and is preferably constituted by at least two half-ridges 26A and 26B projecting radially outwards from the tube 2, positioned on either side of the first slot 22, preferably close to the first face 6C of the ring 6, and facing each other.

The flexible band 25 can thus be moved, e.g. by being deformed elastically, so as to co-operate simultaneously with both half-ridges 26A and 26B in such a manner as to press and hold said half-ridges 26A and 26B against each other and prevent them from moving apart, i.e. prevent the lips 22A and 22B from moving apart, which amounts to locking the tube 6 in its closure configuration.

The flexible band 25 is thus arranged so as to be capable of moving, preferably in reversible manner, between an unlocking position as shown for example in FIGS. 1 and 2, in which said band 25 is substantially parallel to the face 6B, and a locking position in which the band is stretched and hooked at a slant against the ridge 26, as shown in FIG. 3. In order to make the band 25 as easy as possible to manipulate, it is attached to the tube 6 at one point, which point is diametrically opposite the position of the ridge 26.

Advantageously, the band 25 is provided with a pull-tab 27 arranged to enable the band 25 to moved towards the ridge 26 and to co-operate therewith, and/or to enable said band 25 and ridge 26 to be moved apart and separated. For this purpose, the second pull-tab 27 is advantageously in the form of a plane tongue attached to the outer periphery of the flexible band 25 substantially in register with the ridge 26.

The invention thus relates in particular and independently to a surgically implantable ring 1, for surrounding one or more organs constituting a pouch and/or a duct in order to modify the section of the passage in said organ(s), said ring 1, being in the form of a flexible strap 2, 20 extending between first and second ends 3, 4, 40, 400, said flexible strap 2, 20 being provided towards said first and second end 3, 4, 40, 400 with respective male and female closure elements 5, 50, 6, 60, 600 arranged to co-operate in such a manner that the flexible strap 2, 20 forms a closed loop, the locking/unlocking means 21A, 21B, 210A, 210B, 2100A, 2100B being arranged to vary between a locking position in which they prevent said structural discontinuity 20, 200, 2000 from being deformable and an unlocking position in which they substantially release the female closure element 6, 60, 600 from any stress, said locking/unlocking means comprising a flexible band 25 permanently secured to the ring, said band 25 being capable of being moved by being deformed elastically into clamping co-operation with the female element so as to lock the female element in the closure configuration.

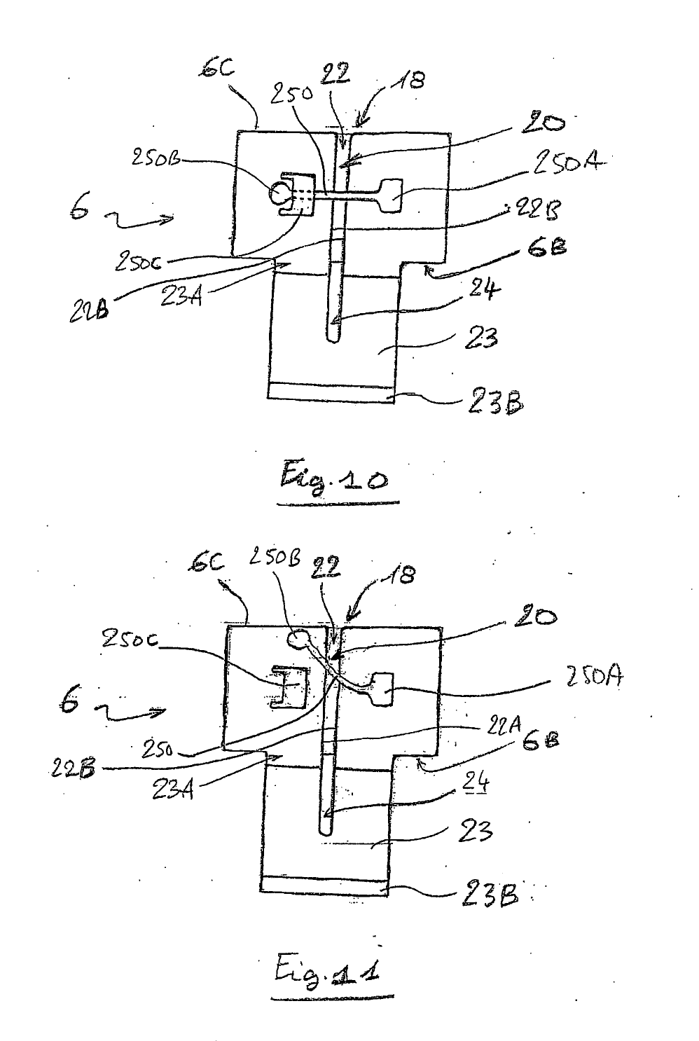

In the variant exemplary embodiment shown in FIGS. 10 and 11, instead of and replacing the band 25 there is provided a catch enabling the lips 22A and 22B to be reversibly connected together. Said catch is in the form of a cord 250 extending lengthwise between a first end 250A secured to the lip 22B and a free second end forming an enlarged head 250B. Said head 250B is arranged to co-operate with a complementary anchor piece 250C secured to the other lip 22A. The cord 250 is preferably resilient and arranged to be capable of varying, preferably in reversible manner, between a locking position shown in FIG. 10 in which said cord 250 is pulled longitudinally to co-operate by resilient return with the anchor piece 250C by the head 250B anchoring against the anchor piece 250C, and secondly an unlocking position (cf. FIG. 11) in which the cord 250 is separated from any interaction with the anchor piece 250C.

The first variant exemplary embodiment of the invention operates as follows.

The surgeon begins by inserting the gastric ring 1 endoscopically into the body of the patient. During this insertion stage, the ring 1 is in developed form, i.e. it is substantially elongate in shape.

The surgeon then holds the flexible strap 2 so that its second end 4 is stationary, e.g. by using the first pull-tab 23, and thereafter causes the rest of the flexible strap 2 to pass around the stomach.

The surgeon then proceeds to close the ring 1 by engaging the antenna 16 followed by the sheath 17, the shape 11, and the first portion 7 into the opening 18 of the tube 6 via the first face 6B of the tube 6. As shown in FIG. 5, the tube 6 presents excellent ability for deforming, in particular when passing the first portion 7 of the male element 5, this ability to deform coming mainly from the first and second inter-communicating slots 22 and 24.

Once the first portion 7 has passed right through the opening 18, 19 of the tube 6, then the tube is in register with the second portion 8 of section that is significantly smaller than the section of the first portion 7. The tube 6 is then no longer subjected to deformation stress and can return automatically into the closure configuration (cf. FIG. 4) because of the elastic nature of the material from which it is made.

The ring 1 is then in the configuration shown in FIG. 2. During this closure stage, care is taken to ensure that the antenna 16, the sheath 17, the shape 11, and also the first portion 7 have all passed through the band 25.

The surgeon then merely needs to take hold of the second pull-tab 27 and pull it so as to bring the resilient band 25 into co-operation with the ridge 26, as shown in FIG. 3. In this locking position, the band 25 presents only residual resilience since its elastic properties have been used to pass it from its unlocking configuration to its locking configuration. The band 25 is thus in a position to exert a firm or even substantially rigid hooping action around the first slot 22 so as to prevent any separation of the lips 22A and 22B.

The ring 1 is thus closed and locked around the stomach of the patient. This closed and locked configuration is particularly stable since the tube 6 is clamped between the first and second shoulders 9, 10 which prevent any axial sliding of said tube 6.

A second exemplary embodiment of the invention is described in greater detail with reference to FIG. 6, this exemplary embodiment differing from the first solely in the shape of the female element 60.

In this second variant exemplary embodiment, the tube 60 is provided like the tube 6 used in the first variant exemplary embodiment with a rectilinear slot 220 formed longitudinally through the entire thickness of its side wall 60A and opening out into first and second faces 60B and 60B between which the tube 60 extends longitudinally.

Advantageously, the tube 60 is provided on either side of said slot 220 with respective half-buttons 28 and 29, said half-buttons 28 and 29 together forming a resulting single complete button 60 when the tube is in the closure configuration, as shown in FIG. 6. Said resulting button 28, 29 is for co-operating with a corresponding buttonhole 31A for locking the ring 60 in the closure configuration, said half-buttons 28, 29 and said buttonhole 31A thus forming the locking/unlocking means 210A, 210B.

Advantageously, the buttonhole 31A is formed by a slot formed in a third pull-tab 31, which tab is attached to the ring 1, preferably close to the first end 3, and more precisely close to the male element 5. The third pull-tab 31 enables the buttonhole 31A to be brought into locking co-operation with the resulting button 28, 29 and/or, where appropriate, enables said buttonhole 31A to be separated from the resulting button 28, 29.

Advantageously, the third pull-tab 31 extends substantially parallel to the plane of the first face 60B of the tube 60, and towards the outside of the ring 1 when it forms a closed loop, as shown in FIG. 6. Preferably, said third pull-tab 31 is itself provided with a fourth pull-tab 32 extending substantially perpendicularly from the third pull-tab 31 so as to be substantially tangential to the ring 1 when it forms a closed loop. Said fourth pull-tab 32 also extends away from the resulting button 28, 29, said fourth tab 32 making it easier to perform opening/closing and locking/unlocking operations on the ring 1.

The operation of the second variant exemplary embodiment is, at least during initial stages, close to the operation of the first variant exemplary embodiment as described above. The main difference is that in the second variant, the tube 60 can open completely into as the first portion 70 passes through. This feature makes it possible to envisage co-operation between male and female elements 50 and 60 not by threading the male element 50 through the female element 60, but by inserting it therein sideways, for example. Thereafter, once the first portion 70 has passed, the ring 60 returns automatically by shape memory to its closure configuration as shown in FIG. 6, thus having the effect of moving the two half-buttons 28 and 29 towards each other and in register so that all that remains to be done is for them to be locked together by interacting with the buttonhole 31A, which the surgeon folds down so as to come into locking engagement with the resulting button 28, 29.

The third variant exemplary embodiment of the invention is described below, as shown in FIGS. 7 and 8. This third variant exemplary embodiment differs from the two preceding variants by the particular shape of its closure member 600 which still forms a tube.

In this variant exemplary embodiment, the tube 600 which extends longitudinally between first and second opposite faces 600B and 600C is provided likewise with a first slot 2200 formed throughout the entire thickness of the side wall 600A of the tube 600, between said first and second faces 600B and 600C. Said first slot 2200 opens out into each of said faces 600B, 600C.

In this third exemplary embodiment, the first slot 2200 comprises at least three individual slots, namely first and second rectilinear individual slots 2200A and 2200B interconnected by a link slot 2200C. Said first individual slot 2200A extends longitudinally in the side wall 600A from the first face 600B to substantially halfway along the distance between the first and second faces 600B and 600C. The second individual slot 2200B is formed longitudinally from the second face 600C to substantially halfway along the distance between said first and second faces 200B and 200C. Finally, the link slot 2200C extends sideways over the periphery of the tube 600 substantially perpendicularly to said first and second individual slots 2200A and 2200B extending each of said individual slots 2200A and 2200B so as to co-operate with them to form a single resulting slot, constituting the first slot 2200.

Thus, in projection, the first slot 2200 is in the form of two staircase steps 2200A, 2200B interconnected by a riser 2200C. The particular shape of the first slot 2200 thus enables two independent flexible arms 33 and 34 to be formed in the tube 600, which arms are suitable for moving in deformation so as to enlarge the opening 180 in order to obtain an enlarged opening 190.

Advantageously, the ring 600 is also provided with first and second locking tabs 35, 36 forming locking means 2100A, 2100B. Said tabs 35, 36 are situated respectively on either side of link slots 2200C, so that each of the tabs 35 and 36 is attached to respective one of the arms 33 and 34.

Each locking tab 35, 36 is also provided with respective hooking and latching means 37A, 37B, 38A, 38B enabling said tabs 35 and 36 to be locked together in position when the tube 600 is in the closure position, in which it is clamped around the second portion 8 of the male element 5.

The locking tabs 35 and 36 extend substantially radially outwards from the tube 600 from the side wall 600A thereof.

Each locking tab 35, 36 is provided with two complementary side tabs 37A, 37B, 38A, 38B secured to the locking tabs 35, 36 in such a manner that each pair of complementary tabs 37A, 37B, 38A, 38B co-operates with the corresponding locking tab 35, 36 associated therewith substantially to form a U-shape. Thus, for the locking tab 36, the limbs of the U-shape are formed respectively by the complementary tabs 38a, 38B, while the web of the U-shape is formed by the locking tab 36 itself. When the tube 600 is in the locking configuration, as shown in FIG. 7, one of the limbs 38A of the U-shape attached to the locking tab 36 is inserted between the limbs 37A, 37B of the U-shape relating to the locking tab 36 attached to the arm 34. The limb 38A is thus interposed between the limbs 37A and 37B, thereby preventing any lateral displacement of the limb 38A, which also comes into abutment against the locking tab 35.

In reciprocal manner, one of the limbs 37A relating to the locking-tab 35 is interposed between the limbs 38A and 38B of the U-shape relating to the locking tab 36.

This particular arrangement thus enables the tube 600 to be locked in its closure configuration. This locking is made even more reliable by the co-operation between the first and second faces 600B, 600C of the tube 600 with the first and second shoulders 9 and 10 of the male element 5.

Thus, the surgical ring 1 in accordance with the invention makes it possible to obtain an excellent compromise firstly between the need to enable it to be closed and secondly the ability to latch the male and female elements together that achieve said closure. The implantable surgical ring 1, in accordance with the invention is also extremely simple to manufacture, using any of the techniques well known to the person skilled in the art, e.g. by molding a silicone type elastomer material. More particularly, the ring 1, can be made as a single piece.

The invention finds one exemplary application in the manufacture and the use of surgical rings for treating obesity. All documents referred to herein are incorporated by reference in their entirety.

1. An implantable surgical ring for surrounding one or more organs having a pouch or a duct, in order to modify the section of the passage in said organ(s), said ring being in the form of a flexible strap extending between first and second ends, said flexible strap being provided towards its first and second ends with respective male and female closure elements arranged to co-operate in such a manner that the flexible strap forms a closed loop, the female closure element permanently forming a closed collar, the ring being characterized in that said female element includes a structural discontinuity in which the deformability of the female element is greater than the deformability of the remainder of the female element.

2. The ring according to claim 1, wherein said female element is arranged to pass reversibly between the closure configuration and the passage configuration.

3. The ring according to claim 1, wherein said ring includes locking/unlocking means for locking/unlocking the female closure element in its closure configuration.

4. The ring according to claim 3, wherein said locking/unlocking means are arranged to vary between a locking position in which they inhibit the deformability of said structural discontinuity, and an unlocking position in which they substantially release the female closure element from any stress.

5. The ring according to claim 1, wherein said male element comprises at least first and second portions, with the section of the first portion being substantially greater than the section of the second portion, said male element being designed to have its first portion threaded through the female element until the female element operates with the second portion, which second portion is interposed between the first portion and the first end of the flexible strap.

6. The ring according to claim 1, wherein said male element is substantially cylindrical in shape, said cylinder being provided with a groove set back from its periphery, said groove forming the second portion.

7. The ring according to claim 1, wherein said female closure element comprises a tube, said tube having a side wall defining said small opening, said side wall being split by at least a first slot, such that said first slot forms said structural discontinuity.

8. The ring according to claim 7, wherein said tube is provided with a first pull-tab arranged to make it easier to move the male element and the tube towards each other into co-operation, and/or easier to separate said male and female elements and to move them apart.

9. The ring according to claim 8, wherein the first pull-tab extends between an attachment end which is secured to the tube and a free end for grasping, said first pull-tab being split longitudinally by a second slot over a fraction only of its length starting from its attachment end, said first pull-tab being attached to the tube in the vicinity of the first slot such that said first slot is extended by the second slot, said first and second slots forming a single resultant slot.

10. The ring according to claim 8, wherein the first pull-tab extends in a direction substantially parallel to the longitudinal direction of the side wall of the tube.

11. The ring according to claim 7 wherein the tube extends longitudinally between first and second opposite faces, the male element being threaded through the female element from the first face towards the second face, the locking/unlocking means comprising a flexible band attached locally to the flexible strap and/or to the tube in such a manner that said band extends substantially in a plane situated in front of the second face, and secondly a latching ridge situated on the periphery of the tube and formed by two half-ridges positioned on either side of the first slot, said band being capable of being brought by elastic deformation into co-operation with said ridge so as to lock the tube in the closure configuration.

12. The ring according to claim 11, wherein the band is provided with a second pull-tab arranged to enable the band to be brought up to and to co-operate with the ridge, and/or to enable the band to be separated from the ridge and moved away therefrom.

13. A ring according to claim 1, further comprising a system for reversibly controlling variation in its internal perimeter, said system comprising an elongate flexible element inserted longitudinally and slidably in the material forming the flexible strap substantially between its first and second ends so as to define a stationary portion secured to the second end and a free portion functionary associated with an actuator mounted on the ring in such a manner that the actuator can cause the elongate flexible element to move reversibly in translation so as to obtain an associated change in the diameter of the ring.

14. The ring according to claim 13, wherein said actuator is arranged on the flexible strap in such a manner as to constitute and/or be included in the first portion of the male element.

15. The ring according to claim 1, wherein said ring constitutes a gastric ring for implanting around the stomach or the esophagus.

16. The ring according to claim 1, wherein said ring is formed by a ring for implanting around the bladder or the urinary tract, or around the gastrointestinal tract, or around blood vessels.

17. The ring according to claim 2, wherein said ring includes locking/unlocking means for locking/unlocking the female closure element in its closure configuration.

18. The ring according to claim 9, wherein said first pull-tab extends in a direction substantially parallel to the longitudinal direction of the side wall of the tube.
